(19) United States
(12) Patent Application Publication
(10) Pub. No.: US 2023/0372531 A1
(43) Pub. Date: Nov. 23, 2023

(54) ENHANCED DELIVERY VEHICLE FOR A TREATING AGENT

(71) Applicant: The Trustees of Columbia University in the City of New York, New York, NY (US)

(72) Inventors: Fatemeh MOMEN-HERAVI, Teaneck, NJ (US); Akiva MINTZ, Paramus, NJ (US)

(21) Appl. No.: 18/365,933

(22) Filed: Aug. 4, 2023

Related U.S. Application Data
(63) Continuation of application No. PCT/US22/15463, filed on Feb. 7, 2022.
(60) Provisional application No. 63/146,214, filed on Feb. 5, 2021.

(51) Int. Cl. A61K 47/69; A61K 45/06

(52) U.S. Cl. CPC A61K 47/6909; A61K 45/06

(57) ABSTRACT

An enhanced delivery vehicle for a treating agent includes a base delivery vehicle, which is adapted for carrying a treating agent for uptake thereof by a target cell, and an enhancing component. The enhancing component may be at least one energy rich substance, at least one agent which increases production of at least one energy rich substance in the target cell, or a combination thereof. The enhancing component enhances the uptake of the base delivery vehicle by the target cell. Non-limiting examples of the base delivery vehicle include exosomes, microvesicles, apoptotic bodies, oncosomes, extracellular vesicles, microparticles, liposomes, and nanoparticles. In one embodiment, the enhancing component is an exosome engineered to be substantially devoid of endogenous nucleic acids. In one embodiment, the enhancing component is at least one energy rich substance including, but not limited to, ATP, NADH, NADPH, FADH2, acetyl CoA, glucose, pyruvate, GTP, CTP, and UTP.

### CROSS-REFERENCE TO RELATED APPLICATIONS

This application is a continuation of international Patent Application No. PCT/US2022/015463, filed Feb. 7, 2022, which claims the benefit of U.S. Provisional Patent Application No. 63/146,214, filed on Feb. 5, 2021, the contents of which are hereby incorporated in their entirety herein.

### TECHNICAL FIELD

The disclosure of the present patent application relates to pharmaceutical treatments, and particularly to an enhanced delivery vehicle for a treating agent which includes an enhancing component for enhancing and increasing the uptake of the delivery vehicle by the target cell.

### BACKGROUND ART

Extracellular vesicles (EVs), including exosomes and microvesicles, are membrane-bounded vesicles secreted by almost all types of cells and are found in biofluids. They naturally carry biomacromolecules, including different RNAs (including mRNAs and microRNAs), DNA, lipids, and proteins, and can efficiently deliver their cargoes to recipient cells, eliciting functions and mediating cellular communications. Different types of EVs, particularly exosomes, can be used for drug delivery.

However, for EVs and exosomes (as well as other types of delivery vehicles) to deliver cargo, they need to be taken up by target cells in an efficient manner, which is one of the limiting steps in the design and manufacture of effective pharmaceutical treatments. Experimental evidence suggests that EV/exosome uptake is accomplished via endocytosis in the majority of cases (and cell fusion in a minority of cases). Inhibiting the endocytosis pathway greatly reduces endosome uptake. The capacity of cells to take up exosomes is greatly attenuated at 4° C., suggesting an energy-dependent non-passive process. In addition, dead fixed cells cannot take up exosomes. Thus, it would be desirable to increase exosome uptake into cells, such as through delivery of additional energetic substances. If such an effect could be achieved, this would enable increased target cell uptake of the therapeutic cargo. Accordingly, enhanced delivery vehicles for treating agents solving the aforementioned problems is desired.

### DISCLOSURE

An enhanced delivery vehicle for a treating agent includes a base delivery vehicle, adapted to carry a treating agent for uptake by a target cell, and an enhancing component. The enhancing component may be at least one energy rich substance; at least one agent which increases production of at least one energy rich substance in the target cell; or a combination thereof. The enhancing component enhances the uptake of the base delivery vehicle by the target cell.

Non-limiting examples of the base delivery vehicle include exosomes, microvesicles, apoptotic bodies, oncosomes, extracellular vesicles, microparticles, liposomes, and nanoparticles. In one embodiment, the enhancing component is an exosome engineered to be substantially devoid of endogenous nucleic acids. In one embodiment, the enhancing component is at least one energy rich substance including, but not limited to, ATP, NADH, NADPH, FADH2, acetyl CoA, glucose, pyruvate, GTP, CTP, and UTP.

The enhanced delivery vehicle may also include additional cargo. Non-limiting examples of such additional cargo include therapeutic agents, diagnostic agents, and a combination thereof. Additional non-limiting examples of additional cargo include, but are not limited to, a nucleic acid, a protein, a polypeptide, a small molecule and combinations thereof.

Further non-limiting examples of additional cargo include a nucleic acid selected from the group consisting of a single-stranded DNA (ssDNA), a double-stranded DNA (dsDNA), a donor/template DNA, a cDNA, a DNA encoding one or more RNAs, a single guide RNA (sgRNA), a guide RNA (gRNA), a prime editing guide RNA (pegRNA), a microRNA (miRNA) inhibitor, a miRNA mimic, a small interfering RNA (siRNA), a small synthetic RNA, a synthetic RNA, a short hairpin RNA (shRNA), a double-stranded RNA (dsRNA), an antisense RNA, an antisense oligonucleotide, a ribozyme, and combinations thereof.

Additional non-limiting examples of additional cargo include, but are not limited to, an endonuclease selected from the group consisting of a zinc finger nuclease (ZFN), a ZFN dimer, a ZFNickase, a transcription activator-like effector nuclease (TALEN), a meganuclease, and an RNA-guided DNA endonuclease. In one embodiment, the additional cargo is an RNA-guided DNA endonuclease, which may be a Clustered Regularly Interspaced Short Palindromic Repeats (CRISPR)-CRISPR associated (Cas) (CRISPR-Cas) system.

In use, a target cell is contacted with the enhanced delivery vehicle described above. These and other features of the present subject matter will become readily apparent upon further review of the following specification.

### BRIEF DESCRIPTION OF THE DRAWINGS

FIG. 1A shows images of fluorescently labeled exosomes devoid of nucleic acids (safeEXO) loaded with ATP using electroporation. Exosomes and controls were subsequently exposed to epithelial cells. Control labeled exosomes were either mock electroporated or co-incubated with ATP without electroporation. The images show significantly increased exosome uptake in ATP-loaded exosomes.

FIG. 1B is a graph of the quantification of the results of FIG. 1A, specifically showing the percent increase of uptake. A greater than 300% increased uptake with ATP-loaded exosomes can be seen.

FIG. 1C is a graph showing the percent increase of uptake of exosomes loaded with ATP in an independent manner using the Exo-Fect™ transfection reagent (provided by System Biosciences, LLC of California). Similar to electroporation, the results demonstrated a greater than 300% increased uptake with ATP-loaded exosomes.

FIG. 2 shows fluorescent cell images for KRAS 4B wild type cells treated with PKH26 (red fluorescent)-labeled safeEXO containing ATP or no ATP as indicated. SafeEXO is a platform that manipulates the exosome creation machinery in producer cells to significantly minimize endogenous RNA loading into exosomes.

FIG. 3 is a graph comparing the level of miR-139-5p for exosomes loaded with ATP, miR-139-5p, GTP, or combinations thereof, and a negative control.

Similar reference characters denote corresponding features consistently throughout the attached drawings.

### DESCRIPTION OF EMBODIMENTS

The enhanced delivery vehicle for a treating agent includes a base delivery vehicle, which is adapted for carrying a treating agent for uptake thereof by a target cell, and an enhancing component. The enhancing component may be at least one energy rich substance, at least one agent which increases production of at least one energy rich substance in the target cell, or a combination thereof. The enhancing component enhances the uptake of the base delivery vehicle by the target cell.

Non-limiting examples of the base delivery vehicle include exosomes, microvesicles, apoptotic bodies, oncosomes, extracellular vesicles, microparticles, liposomes, and nanoparticles. In one embodiment, the enhancing component is an exosome engineered to be substantially devoid of endogenous nucleic acids. In one embodiment, the enhancing component is at least one energy rich substance including, but not limited to, ATP, NADH, NADPH, FADH2, acetyl CoA, glucose, pyruvate, GTP, CTP, and UTP.

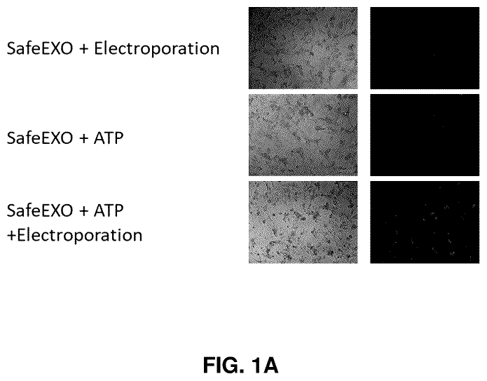

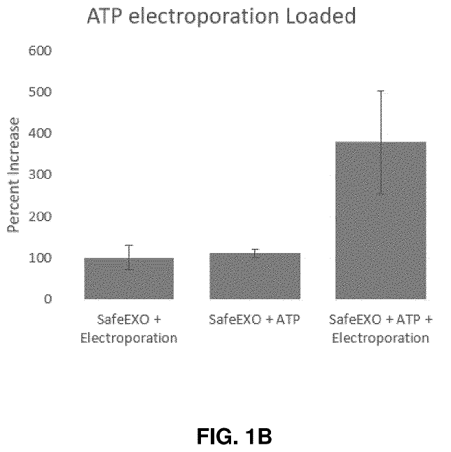

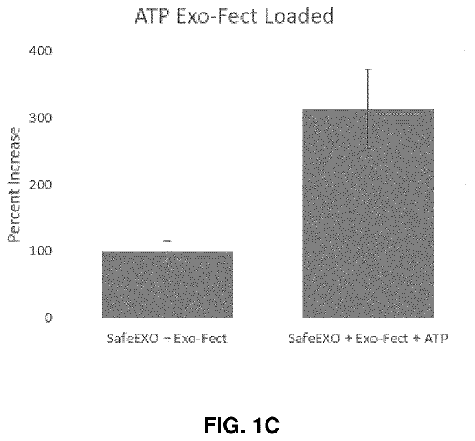

The efficacy of ATP-loaded exosomes taken up by target cells, in particular, will be described in detail below. As will be discussed in detail below, loading exosomes with ATP using two independent methods, either electroporation or a transfection reagent (Exo-Fect™, provided by System Biosciences, LLC of California), showed a significantly increased target cell uptake of the exosomes. ATP-loaded exosomes loaded using electroporation showed a 380% increase in uptake. ATP-loaded exosomes loaded using Exo-Fect™ showed a 313% increase in uptake (See Example 1 below and FIGS. 1B and 1C). This data confirms that co-delivering ATP or equivalents (NADH, NADPH, FADH2, acetyl CoA, glucose, pyruvate, etc.) as well as agents which increase the production or amount of at least one energy rich substance in the target cell can enhance exosome target cell uptake.

### Delivery Vehicles

The delivery vehicles which can be used in the compositions and methods disclosed herein include any delivery vehicle that is capable of entering a cell. These delivery vehicles may be synthetic or natural delivery vehicles and include, but are not limited to, exosomes, microvesicles, apoptotic bodies, oncosomes, extracellular vesicles, microparticles, liposomes, and nanoparticles.

### Extracellular Vesicles and Exosomes

Extracellular vesicles (EVs) are membrane-enclosed vesicles released by cells. Their primary constituents are lipids, proteins, and nucleic acids. EVs are typically composed of a lipid-protein bilayer encapsulating an aqueous core comprising nucleic acids and soluble proteins. Extracellular vesicles include, but are not limited to, exosomes, shedding vesicles, microvesicles, small vesicles, large vesicles, microparticles, oncosomes and apoptotic bodies, based on their diameter, cellular origin and formation mechanism. Non-limiting types of extracellular vesicles include circulating extracellular vesicles, beta cell extracellular vesicles, islet cell extracellular vesicles, exosomes and apoptotic bodies, and combinations thereof.

Exosomes are formed by inward budding of late endosomes forming multivesicular bodies (MVB) which then fuse with the limiting membrane of the cell concomitantly releasing the exosomes. Exosomes generally are nanosized (50-150 nm) membrane bounded vesicles secreted by almost all types of cells, and are often found in biofluids.

Shedding vesicles are formed by outward budding of the limiting cell membrane followed by fusion.

When a cell undergoes apoptosis, the cell disintegrates and divides its cellular content in different membrane-enclosed vesicles termed apoptotic bodies.

Large extracellular vesicles can range from about 5 μm to about 12 μm in diameter. Apoptotic bodies can range from about 1 μm to about 5 μm in diameter. Microvesicles can range from about 100 nm to about 1 μm in diameter. Exosomes can range from about 30 nm to about 150 nm, from about 30 nm to about 100 nm, or from about 50 nm to about 150 nm in diameter or from about 50 nm to about 200 nm.

Extracellular vesicles, without limitation such as exosomes, may be isolated or derived from bone marrow, red blood cells, tumor cells, immune cells, epithelial cells, fibroblasts, stem cells, or from B cells, T cells, monocytes, or macrophages. In one embodiment, extracellular vesicles to be taken up by a specific type of cells (e.g., monocytes/macrophages) are isolated or derived from the same type of cells (e.g., monocytes/macrophages).

Extracellular vesicles may be isolated or derived from a body fluid. For example, the body fluids can include, but are not limited to, serum, plasma, blood, whole blood and derivatives thereof, urine, tears, saliva, sweat, cerebrospinal fluid (CSF), oral mucus, vaginal mucus, seminal plasma, semen, prostatic fluid, excreta, ascites, lymph, bile, breast milk and amniotic fluid. Extracellular vesicles may also be isolated or derived from cultured cells.

Methods for isolating extracellular vesicles include size separation methods, such as centrifugation. In one embodiment, isolating various components of extracellular vesicles may be accomplished by an isolation method including sequential centrifugation. The method may include centrifuging a sample at 800 g for a desired amount of time, collecting the pellet containing cells and cellular debris and (first) supernatant, centrifuging the (first) supernatant at 2,000 g for a desired time, and collecting the pellet containing large extracellular vesicles and apoptotic bodies and (second) supernatant. The sequential centrifugation method may further include centrifuging the (second) supernatant at 10,000 g, and collecting the pellet containing microvesicles and (third) supernatant. The sequential centrifugation method may further include centrifuging the (third) supernatant at 100,000 g, and collecting the pellet containing exosomes (ranging from about 30 nm to about 200 nm in diameter) and (fourth) supernatant.

It should be understood that any suitable method for isolating extracellular vesicles may be used, including, but not limited to, ultracentrifugation (sedimentation based on size and density), size exclusion filtration, immune affinity isolation (antibody against specific EVs surface proteins), microfluidic techniques (trapping EVs in micro channels), polymeric precipitation methods (reduce EV solubility and drive precipitation by dissolving polymers), size exclusion chromatography (separation based on molecular weight and size), sieving methods (deriving filtration by pressure or electrophoresis), and porous structures (capturing EVs through porous microstructures based on ciliated micropillar structure).

A sequential centrifugation method can further include washing each of the pellets including the extracellular vesicles (e.g., large extracellular vesicles and apoptotic bodies, microvesicles, and exosomes), such as in phosphate-buffered saline, followed by centrifugation at the appropriate gravitational force, and collection of the pellet containing the extracellular vesicles. Isolation, purity, concentration, size, size distribution, and combinations thereof of the extracellular vesicles following each centrifugation step may be confirmed using methods such as nanoparticle tracking, transmission electron microscopy, immunoblotting, and combinations thereof.

Nanoparticle tracking (NTA) to analyze extracellular vesicles, such as for concentration and size, may be performed by dynamic light scattering using commercially available instruments such as certain ZetaView® brand instruments (commercially available from Particle Metrix of Meerbusch, Germany). Following isolation, the method can further include detecting an extracellular vesicle marker.

Methods for isolating extracellular vesicles also include using commercially available reagents such as, for example, the ExoQuick-TC™ brand reagent (commercially available from System Biosciences of Palo Alto, California).

Exosomes are small vesicular bodies that are secreted from cells into the cellular microenvironment and biofluids, and can enter both neighboring cells and the systemic circulation. Exosomes are actively assembled from intracellular multivesicular bodies (MVBs) by the endosomal sorting complex required for transport (ESCRT) machinery. Exosomes contain various molecular constituents of their cell of origin, including, but not limited to, proteins, RNA (such as mRNA, miRNA, etc.), lipids and DNA.

Exosomes may be isolated by any suitable techniques, including ultracentrifugation, micro-filtration, size-exclusion chromatography, etc., or a combination thereof. Exosomes can be isolated using a combination of techniques based on both physical (e.g., size and density) and biochemical parameters (e.g., presence/absence of certain proteins involved in their biogenesis). In certain embodiments, exosomes are isolated using a kit. In one embodiment, exosomes are isolated using the Total Exosome Isolation Kit and/or the Total Exosome Isolation Reagent available from Invitrogen®.

Following isolation, the method can further include detecting an extracellular vesicle marker associated with the extracellular vesicle. Extracellular vesicle or exosome markers include CD9, CD63, CD81, LAPM1, TSG101, and combinations thereof. Exosomes naturally carry biomacromolecules, including different RNAs (mRNAs, miRs), DNA, lipids, and proteins, and can efficiently deliver their cargoes to recipient cells, eliciting functions and mediating cellular communications. Previous work has shown the advantages of using exosomes/extracellular vesicles for drug delivery, including: 1) exosomes are small and have a high efficiency for delivery due to their similarity to cell membranes; 2) exosomes are biocompatible, non-immunogenic, and non-toxic, even in repeated in vivo injections; 3) exosomes are stable even after several freeze and thaw cycles, and their lipid bilayer protects the protein and RNA cargoes from enzymes such as proteases and RNases; 4) exosomes have a slightly negative zeta potential, leading to long circulation; and 5) exosomes also exhibit an increased capacity to escape degradation or clearance by the immune system.

One challenge with using exosomes for delivery of RNAi, RISC complex proteins or nucleic acid stabilizing proteins is that they contain endogenous RNA that might be harmful to the patient, such as by promoting tumorigenesis, exacerbating diseases such as cancer. Thus, delivering RISC complex proteins together with potentially harmful endogenous RNA may intensify adverse events. We therefore produced our engineered safeEXO (ES-exo) platform that manipulates the exosome creation machinery in producer cells to significantly minimize endogenous RNA loading into exosomes. Engineered exosomes or extracellular vesicles substantially devoid of endogenous nucleic acids can be prepared as described in PCT application no. PCT/US2020/047894, which is hereby incorporated by reference in its entirety.

Previous experiments have shown that exosomes can functionally deliver miR inhibitors, RNAis, and miRs. Exosomes were able to successfully deliver miR-155 analogue to the liver and hepatocytes of miR-155 KO mice, and can restore the level to up to 50% of wildtype mice. We have optimized the protocol of loading exosomes with miRs, miR inhibitors, and siRNAs. Repeated injection of exosomes did not induce any adverse effects, cytotoxicity, and immunogenicity in vivo and in vitro.

We have optimized the protocol of loading exosomes with miRs, miR inhibitors, and siRNAs and used that for mechanistic studies in our previous studies. Repeated injection of exosomes did not induce any adverse effects, cytotoxicity, and immunogenicity in vivo and in vitro. Furthermore, we have demonstrated that we can use exosomes to deliver the necessary components to perform CRISPR applications on target cells. For example, we developed an exosome platform that endogenously expresses Cas9 (CRISPR associated protein 9) and can be used to edit cellular DNA when loaded with specific sgRNA.

### Liposomes and Nanoparticles

The formation and use of liposomes are generally known to those having ordinary skill in the art. Recently, liposomes were developed with improved serum stability and circulation half-times, as discussed in U.S. Pat. No. 5,741,516, which is hereby incorporated by reference in its entirety. Further, various methods of liposome and liposome-like preparations as potential drug carriers have been described, such as discussed in U.S. Pat. Nos. 5,567,434; 5,552,157; 5,565,213; 5,738,868; and 5,795,587, each of which is hereby incorporated by reference in its entirety.

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also referred to as “multilamellar vesicles” or MLVs). MLVs generally have diameters of from about 25 nm to about 4 μm. Sonication of MLVs results in the formation of small unilamellar vesicles (SUVs) with diameters in the range of 200 Å to 500 Å, containing an aqueous solution in the core.

Alternatively, nanocapsule formulations may be used. Nanocapsules can generally entrap substances in a stable and reproducible way. Nanoparticles provide a colloidal carrier system that has been shown to improve the efficacy of an encapsulated drug by prolonging the serum half-life. Polyalkylcyanoacrylates (PACAs) nanoparticles are a polymer colloidal drug delivery system that is in clinical development. Biodegradable poly (hydroxyl acids), such as the copolymers of poly (lactic acid) (PLA) and poly (lactic-co-glycolide) (PLGA) are being extensively used in biomedical applications and have received FDA approval for certain clinical applications.

In addition, nanoparticles have many desirable carrier features, including: 1) that the agent to be encapsulated may comprise a reasonably high weight fraction (loading) of the total carrier system; 2) that the amount of agent used in the first step of the encapsulation process is incorporated into the final carrier (entrapment efficiency) at a reasonably high level; 3) that the carrier typically may be freeze-dried and reconstituted in solution without aggregation; 4) that the carrier typically is biodegradable; 5) that the carrier system can be of small size; and 6) that the carrier may enhance the particles' persistence.

Nanoparticles may be synthesized using virtually any biodegradable shell known in the art. Such polymers are biocompatible and biodegradable and are subject to modifications that desirably increase the photochemical efficacy and circulation lifetime of the nanoparticle. In one embodiment, the polymer is modified with a terminal carboxylic acid group (COOH) to increase the negative charge of the particle and thus limit the interaction with negatively charged nucleic acids. Nanoparticles may also be modified with polyethylene glycol (PEG), which increases the half-life and stability of the particles in circulation. Alternatively, the COOH group may be converted to an N-hydroxysuccinimide (NHS) ester for covalent conjugation to amine-modified compounds.

### Energy Rich Substances

The delivery vehicles disclosed herein are loaded with an energy rich substance which provides the energy necessary for a cell to uptake the delivery vehicle. Energy rich substances for use in the composition and methods disclosed herein include, but are not limited to, ATP, NADH, NADPH, FADH2, acetyl CoA, glucose, pyruvate, GTP, CTP, UTP, and combinations thereof.

### Agents May Increase Production or Amount of Energy Rich Substances in Target Cell

The delivery vehicles disclosed herein can also be loaded with agents that increase the production or amount of an energy rich substance in the target cell, in turn providing the energy necessary for the cell to uptake the delivery vehicle. Agents are defined herein as substances that produce, or are capable of producing, the desired effect and would include, but are not limited to, chemicals, pharmaceuticals, biologics, small organic molecules, antibodies, nucleic acids, peptides, and proteins.

Agents which can be used in the compositions and methods disclosed herein include, but are not limited to, siRNAs for Apyrase inhibitor, ATPase inhibitor targeting ATP or ADP carrier proteins, activation or delivery of AMP-activated protein kinase. Energy rich substances which would be increased by the agents include, but are not limited to, ATP, NADH, NADPH, FADH2, acetyl CoA, glucose, pyruvate, GTP, CTP, and UTP.

### Additional Cargo or Payload

In some embodiments, the delivery vehicle comprising the at least one energy rich substance or the at least one agent which increases the production or amount of at least one energy rich substance, further comprises additional cargo or payload to be delivered to the cell. The cargo or payload of the present delivery vehicles may include, but is not limited to, therapeutic and diagnostic agents. The cargo or payload of the present delivery vehicles may include, but is not limited to, a nucleic acid (DNA, RNA, etc.), a protein or polypeptide, or a small molecule, or combinations thereof. Proteins or polypeptides include, but are not limited to, a nuclease (an endonuclease or an exonuclease), an antibody or a fragment thereof, or combinations thereof.

Nucleic acids include DNA or RNA. DNA includes a single-stranded DNA (ssDNA), a double-stranded DNA (dsDNA), a donor/template DNA, a DNA encoding one or more RNAs, cDNA, or combinations thereof. RNA includes a single guide RNA (sgRNA), a guide RNA (gRNA), prime editing guide RNA (pegRNA), a microRNA (miRNA) inhibitor, a miRNA mimic, a small interfering RNA (siRNA), small synthetic RNA, synthetic RNA, an antisense oligonucleotide, a short hairpin RNA (shRNA), a double-stranded RNA (dsRNA), an antisense RNA, a ribozyme, or combinations thereof. The cargo or payload may also be a DNA digesting agent.

In certain embodiments, the nucleic acid (e.g., gRNA, sgRNA, etc.) may contain a nuclear localization signal (NLS) which may increase the efficacy of editing in the nucleus. An NLS is an amino acid sequence that tags a protein for import into the cell nucleus by nuclear transport. In one embodiment, the NLS is the Chelsky sequence motif of K-K/R-x-K/R (Lys-Lys/Arg-x-Lys/Arg) which leads to nuclear localization via importin a. In another embodiment, the NLS is a bipartite NLS of nucleoplasmin, or other NLS sequences such as c-Myc (PAAKRVKLD) and TUS-protein (KLKIKRPVK).

As used herein, the term “small molecules” encompasses molecules other than proteins or nucleic acids without strict regard to size. Non-limiting examples of small molecules that may be used according to the methods and compositions of the present invention include, but are not limited to, small organic molecules, peptide-like molecules, peptidomimetics, carbohydrates, lipids or other organic (carbon containing) or inorganic molecules.

The term “DNA digesting agent” refers to an agent that is capable of cleaving bonds (e.g., phosphodiester bonds) between the nucleotide subunits of nucleic acids. In one embodiment, the DNA digesting agent is a nuclease. Nucleases are enzymes that hydrolyze nucleic acids. Nucleases may be classified as endonucleases or exonucleases. An endonuclease is any of a group of enzymes that catalyze the hydrolysis of bonds between nucleic acids in the interior of a DNA or RNA molecule. An exonuclease is any of a group of enzymes that catalyze the hydrolysis of single nucleotides from the end of a DNA or RNA chain. Nucleases may also be classified based on whether they specifically digest DNA or RNA. A nuclease that specifically catalyzes the hydrolysis of DNA may be referred to as a deoxyribonuclease or DNase, whereas a nuclease that specifically catalyzes the hydrolysis of RNA may be referred to as a ribonuclease or an RNase. Some nucleases are specific to either single-stranded or double-stranded nucleic acid sequences. Some enzymes have both exonuclease and endonuclease properties. In addition, some enzymes are able to digest both DNA and RNA sequences.

### Endonucleases

Non-limiting examples of the endonucleases include a zinc finger nuclease (ZFN), a ZFN dimer, a ZFNickase, a transcription activator-like effector nuclease (TALEN), a meganuclease, or an RNA-guided DNA endonuclease (e.g., CRISPR/Cas systems). Meganucleases include endonucleases in the LAGLIDADG and PI-Sce family.

TALENs are composed of a TAL effector domain that binds to a specific nucleotide sequence and an endonuclease domain that catalyzes a double strand break at the target site. Sequence-specific endonucleases may be modular in nature, and DNA binding specificity is obtained by arranging one or more modules. ZFNs can be composed of two or more (e.g., 2-8, 3-6, 6-8, or more) sequence-specific DNA binding domains (e.g., zinc finger domains) fused to an effector endonuclease domain (e.g., the FokI endonuclease).

The sequence-specific endonuclease of the methods and compositions described herein can be engineered, chimeric, or isolated from an organism. Endonucleases can be engineered to recognize a specific DNA sequence, by, e.g., mutagenesis. Combinatorial assembly is a method where protein subunits form different enzymes and can be associated or fused. These two approaches (i.e., mutagenesis and combinatorial assembly) may be combined to produce an engineered endonuclease with a desired DNA recognition sequence. The sequence-specific nuclease can be in the form of a protein or in the form of a nucleic acid encoding the sequence-specific nuclease, such as an mRNA or a cDNA.

### CRISPR Systems

The CRISPR (Clustered Regularly Interspaced Short Palindromic Repeats) technology exploits RNA-guided DNA-binding and sequence-specific cleavage of target DNA. A guide RNA (gRNA) is complementary to a target DNA sequence. The guide RNA/Cas combination confers site specificity to the nuclease. A single guide RNA (sgRNA) contains about 20 nucleotides that are complementary to a target genomic DNA sequence and a constant RNA scaffold region. The Cas (CRISPR-associated) protein binds to the guide RNA (gRNA) or sgRNA and the target DNA to which the gRNA or sgRNA binds and introduces a double-strand break.

In addition to a sequence that binds to a target nucleic acid, in some embodiments, the gRNA also comprises a scaffold sequence. Expression of a gRNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gRNA may be referred to as a single guide RNA (sgRNA).

Cleavage of a gene region may comprise cleaving one or two strands at the location of the target sequence by the Cas enzyme. In one embodiment, such cleavage can result in decreased transcription of a target gene. In another embodiment, the cleavage can further comprise repairing the cleaved target polynucleotide by homologous recombination with an exogenous template or donor DNA, where the repair results in an insertion, deletion, or substitution of one or more nucleotides of the target polynucleotide.

The terms “gRNA,” “guide RNA” and “CRISPR guide sequence” may be used interchangeably and refer to a nucleic acid comprising a sequence that determines the specificity of a Cas DNA binding protein of a CRISPR/Cas system. A gRNA hybridizes to (complementary to, partially or completely) a target nucleic acid sequence in the genome of a host cell. The gRNA or portion thereof that hybridizes to the target nucleic acid may be between 15-25 nucleotides, 18-22 nucleotides, or 19-21 nucleotides in length. In some embodiments, the gRNA sequence that hybridizes to the target nucleic acid is 15, 16, 17, 18, 19, 20, 21, 22, 23, 24, or 25 nucleotides in length. In some embodiments, the gRNA sequence that hybridizes to the target nucleic acid is between 10-30, or between 15-25, nucleotides in length.

As used herein, a “scaffold sequence,” also referred to as a tracrRNA (trans-activating CRISPR RNA), refers to a nucleic acid sequence that recruits a Cas endonuclease to a target nucleic acid bound (hybridized) to a complementary gRNA sequence. Any scaffold sequence that comprises at least one stem loop structure and recruits an endonuclease may be used in the genetic elements and vectors described herein.

In some embodiments, the gRNA sequence does not comprise a scaffold sequence, and a scaffold sequence is expressed as a separate transcript. In such embodiments, the gRNA sequence further comprises an additional sequence that is complementary to a portion of the scaffold sequence and functions to bind (hybridize) the scaffold sequence and recruit the endonuclease to the target nucleic acid.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid. (See also U.S. Pat. No. 8,697,359, which is hereby incorporated by reference for its teaching of complementarity of a gRNA sequence with a target polynucleotide sequence.)

A gRNA can have a length ranging from about 12 nucleotides to about 100 nucleotides. For example, gRNA can have a length ranging from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the first segment (e.g., crRNA) can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. A gRNA can have fewer than 12 nucleotides or greater than 100 nucleotides.

sgRNA(s) can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, sgRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

The present delivery vehicle may contain one, two or more sgRNAs targeting the DNA encoding one or more proteins or polypeptides. In one embodiment, a Cas protein may be a functional derivative of a naturally occurring Cas protein. In certain embodiments, the Cas enzyme comprises one or more mutations. The Cas enzyme may be a type II, type I, type III, type IV or type V CRISPR system enzyme. In some embodiments, the Cas enzyme is a Cas9 enzyme (also known as Cas5, Csn1, or Csx12). Cas9 may be wild-type or mutant. In some embodiments, the endonuclease is a Cas9 homolog or ortholog. Cas9 may be any variant disclosed in U.S. Patent Publication No. US 2014/0068797 A1, which is hereby incorporated by reference. Cas9 may be Type II-A, Type II-B, or Type II-C.

In some embodiments, the Cas9 is a modified form or a variant of the wild-type Cas9. In certain embodiments, the modified form of the Cas9 protein comprises an amino acid change (e.g., deletion, insertion, or substitution) that reduces the naturally occurring nuclease activity of the Cas9 protein. In certain embodiments, the modified form of the Cas9 protein has less than 50%, less than 40%, less than 30%, less than 20%, less than 10%, less than 5%, or less than 1% of the nuclease activity of the corresponding wild-type Cas9 protein. In some cases, the modified form of the Cas9 protein has no substantial nuclease activity.

Cas9 may be from various species. Non-limiting examples of the Cas9 enzyme include, but are not limited to, Cas9 derived from Streptococcus pyogenes (S. pyogenes), Streptococcus pneumoniae (S. pneumoniae), Staphylococcus aureus, Neisseria meningitidis, Streptococcus thermophilus (S. thermophilus), or Treponema denticola. The Cas enzyme may also be derived from Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma or Campylobacter.

In certain embodiments, the Cas enzyme is Cas9, Cpf1, C2c1, C2c2, C2c3, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, orthologs thereof, or modified versions thereof. In one embodiment, the Cas enzyme is Cas9.

Cas9 harbors two independent nuclease domains homologous to HNH and RuvC endonucleases, and by mutating either of the two domains, the Cas9 protein can be converted to a nickase that introduces single-strand breaks. It is contemplated that the methods and compositions of the present disclosure can be used with the single- or double-strand-inducing version of Cas9, as well as with other RNA-guided DNA nucleases, such as other bacterial Cas9-like systems. The sequence-specific nuclease of the present methods and compositions described herein can be engineered, chimeric, or isolated from an organism.

In some embodiments, the nucleotide sequence encoding the Cas9 endonuclease is further modified to alter the activity of the protein. In some embodiments, the Cas9 endonuclease is a catalytically inactive Cas9. For example, dCas9 contains mutations of catalytically active residues (D10 and H840) and does not have nuclease activity.

Alternatively, or in addition thereto, the Cas9 endonuclease may be fused to another protein or portion thereof. In some embodiments, dCas9 is fused to a repressor domain, such as a KRAB domain. In some embodiments, such dCas9 fusion proteins are used with the constructs described herein for multiplexed gene repression (e.g. CRISPR interference (CRISPRi)). In some embodiments, dCas9 is fused to an activator domain, such as VP64 or VPR. In some embodiments, such dCas9 fusion proteins are used with the constructs described herein for gene activation (e.g., CRISPR activation (CRISPRa)). In some embodiments, dCas9 is fused to an epigenetic modulating domain, such as a histone demethylase domain or a histone acetyltransferase domain. In some embodiments, dCas9 is fused to a LSD1 or p300, or a portion thereof. In some embodiments, the dCas9 fusion is used for CRISPR-based epigenetic modulation. In some embodiments, dCas9 or Cas9 is fused to a Fok1 nuclease domain. In some embodiments, Cas9 or dCas9 fused to a Fok1 nuclease domain is used for genome editing. In some embodiments, Cas9 or dCas9 is fused to a fluorescent protein (e.g., GFP, RFP, mCherry, etc.). In some embodiments, Cas9/dCas9 proteins fused to fluorescent proteins are used for labeling and/or visualization of genomic loci or identifying cells expressing the Cas endonuclease.

Differential gene expression can be achieved by modifying the efficiency of gRNA base-pairing to the target sequence. Modulating this efficiency may be used to create an allelic series for any given gene, creating a collection of hypomorphs and hypermorphs. These collections can be used to probe any genetic investigation. For hypomorphs, this allows the incremental reduction of gene function as opposed to the binary nature of gene knockouts.

CRISPR interference (CRISPRi) or CRISPR activation (CRISPRa) may be used in the present systems and methods. CRISPRi is a transcriptional interference technique that allows for sequence-specific repression of gene expression and/or epigenetic modifications in cells. CRISPRi regulates gene expression primarily on the transcriptional level. CRISPRi can sterically repress transcription, e.g., by blocking transcriptional initiation or elongation. The target sequence may be the promoter and/or exonic sequences (such as the non-template strand and/or the template strand), and/or introns. Specific gene repression by the CRISPRi system can be transferred through bacterial conjugation. CRISPRi can also repress transcription via an effector domain. Fusing a repressor domain to a catalytically inactive Cas enzyme, e.g., dead Cas9 (dCas9), may further repress transcription. For example, the Krüppel associated box (KRAB) domain can be fused to dCas9 to repress transcription of the target gene.

CRISPRa utilizes the CRISPR technique to allow for sequence-specific activation of gene expression and/or epigenetic modifications in cells. For example, a catalytically inactive Cas enzyme, e.g., dCas9, may be used to activate genes when fused to transcription activating factors. These factors include, but are not limited to, subunits of RNA Polymerase II and traditional transcription factors, such as VP16, VP64, VPR etc.

The Cas endonuclease may be a Cpf1 nuclease. In some embodiments, the host cell expresses a Cpf1 nuclease derived from Provetella spp. or Francisella spp. Prime editing CRISPR may also be used in the present systems and methods. The guide RNA used in prime editing CRISPR, called prime editing guide RNA (pegRNA), is substantially larger than standard sgRNAs commonly used for CRISPR gene editing (>100 nt vs. 20 nt). The pegRNA is a sgRNA with a primer binding sequence (PBS) and the template containing the desired RNA sequence added at the 3′ end. Together, they form the PE:pegRNA complex, which is used to mediate genome editing within the cell.

First, an engineered prime editing guide RNA (pegRNA) that both specifies the target site and contains the desired edit(s) engages the prime editor protein. This primer editor protein consists of a Cas9 nickase fused to a reverse transcriptase. The Cas9 nickase part of the protein is guided to the DNA target site by the pegRNA. After nicking by Cas9, the reverse transcriptase domain uses the pegRNA to template reverse transcription of the desired edit, directly polymerizing DNA onto the nicked target DNA strand. The edited DNA strand replaces the original DNA strand, creating a heteroduplex containing one edited strand and one unedited strand. Lastly, the editor guides resolution of the heteroduplex to favor copying the edit onto the unedited strand, completing the process. The Cas9 nickase can be fused to the NI-MIN reverse transcriptase (RT) including mutated versions to create the prime editor (PE) (PE1 and PE2).

Once the prime editor incorporates the edit into one strand, there is a mismatch between the original sequence on one strand and the edited sequence on the other strand. To guide heteroduplex resolution to favor the edit, the non-edited strand is nicked causing the cell to remake that strand using the edited strand as the template. A third prime editing system called PE3 does this by including an additional sgRNA. Using this sgRNA, the prime editor nicks the unedited strand away from the initial nick site (to avoid creating a double strand break), increasing editing efficiencies 2- to 3-fold with indel frequencies between 1-10%.

The other important component of prime editing is the prime editing guide RNA (pegRNA). The pegRNA is a guide RNA that also encodes the RT template, which includes the desired edit and homology to the genomic DNA locus. Sequence complementary to the nicked genomic DNA strand serves as a primer binding site (PBS). This PBS sequence hybridizes to the target site and serves as the point of initiation for reverse transcription. To optimize pegRNAs, extending the pegRNA primer binding site to at least eight nucleotides enables more efficient prime editing. The pegRNA may be between 50-500 nucleotides in length.

### Inhibitory Nucleic Acids

In certain embodiments, the cargo or payload may be an inhibitory nucleic acid or polynucleotide that reduces expression of a target gene. Thus, the polynucleotide specifically targets a nucleotide sequence encoding a target protein or polypeptide. The nucleic acid target of the polynucleotides may be any location within the gene or transcript of the target protein or polypeptide.

The inhibitory nucleic acids may be RNA interference or RNAi, an antisense RNA, a ribozyme, or combinations thereof. “RNA interference”, or “RNAi” is a form of post-transcriptional gene silencing (“PTGS”), and comprises the introduction of, e.g., double-stranded RNA into cells. The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. RNAi can work in human cells if the RNA strands are provided as pre-sized duplexes of about 19 nucleotide pairs, and RNAi worked particularly well with small unpaired 3′ extensions on the end of each strand. RNAi may be small interfering RNA or siRNAs, a small hairpin RNA or shRNAs, microRNA or miRNAs, a double-stranded RNA (dsRNA), etc.

The cargo or payload may be a short RNA molecule, such as a short interfering RNA (siRNA), a small temporal RNA (stRNA), and a micro-RNA (miRNA). Short interfering RNAs silence genes through an mRNA degradation pathway, while stRNAs and miRNAs are approximately 21 or 22 nt RNAs that are processed from endogenously encoded hairpin-structured precursors, and function to silence genes via translational repression. Alternatively, a polynucleotide encoding an siRNA or shRNA may be used.

The inhibitory nucleic acids may be an antisense nucleic acid sequence that is complementary to a target region within the mRNA of a target protein or polypeptide. The antisense polynucleotide may bind to the target region and inhibit translation. The antisense oligonucleotide may be DNA or RNA or comprise synthetic analogs of ribo-deoxynucleotides. An antisense oligonucleotide can be, for example, about 7, 10, 15, 20, 25, 30, 35, 40, 45, 50, 55, 60, 65, 70, 75, 80, or more nucleotides in length. The cargo or payload may be a ribozyme. Ribozymes may be chemically synthesized and structurally modified to increase their stability and catalytic activity using methods well known in the art.

### Antibodies

The cargo or payload may be an antibody or a fragment (e.g., an antigen-binding portion) thereof. The antibody or antigen-binding portion thereof may be the following: (a) a whole immunoglobulin molecule; (b) a single-chain variable fragment (scFv); (c) a Fab fragment; (d) an F(ab′)2; and (e) a disulfide linked Fv. The antibody or antigen-binding portion thereof may be monoclonal, polyclonal, chimeric and humanized. The antibodies may be murine, rabbit or human/humanized antibodies.

### Donor/Template Polynucleotides

The cargo or payload may also contain a donor/template polynucleotide (e.g., DNA). The donor/template polynucleotide may be a single-stranded donor DNA or a double-stranded donor DNA. A DNA double-strand break at a defined site in the genome (e.g., produced by a Cas-family nuclease) may or may not be repaired by the DNA repair machinery. The most involved DNA repair pathways are non-homologous end joining (NHEJ) and homology directed repair (HDR). NHEJ usually leads to a small insertion or deletion and thus gene knockout. NHEJ can be exploited for gene knockout by the introduction of a premature STOP-codon or frame shift of genetic reading. HDR with a donor/template polynucleotide can lead to insertion of a desired sequence of DNA. Gene correction, epigenetic modulation, and knock-in can be obtained through HDR, by addition of a donor/template DNA to the machinery which leads to repair complementary to the provided template.

Depending on the desired genetic manipulation, various components of CRISPR/Cas may be delivered as a cargo or payload by the present engineered exosome or extracellular vesicle: (a) a minimal Cas/gRNA pair for gene disruption/mutation, (b) Cas/gRNA and a donor/template DNA for gene correction, (c) Cas/gRNA and a desired gene for gene insertion (e.g., a donor/template DNA), or (d) Cas9 and two gRNAs for the complete deletion of a gene (or a portion of a gene).

In one embodiment, the donor/template polynucleotide may result in the expression of a corrected gene, which can restore or correct the function of the disease-related gene or fragment after the deletion/mutation/truncation of endogenous gene(s) or fragments. In one embodiment, the cargo or payload comprise a gRNA and a single-stranded donor/template DNA. The gRNA is designed to be within 10 nucleotides of HDR. The 5′ and 3′ homology arms are about 30-40 nucleotides. Each end of the donor/template DNA may contain two phosphonothioates to increase HDR efficacy.

In certain embodiments, the cargo or payload may also include a desired gene for gene insertion. In one embodiment, the desired gene for gene insertion is a codon-modified polynucleotide for a gene of interest. In one embodiment, the donor/template polynucleotide is codon modified to be unrecognizable by the DNA digesting agent (e.g., gRNA or sgRNA). Such donor sequence may encode at least a functional fragment of the protein lacking or deficient in the cell.

Alternatively, if the present composition and method deletes, destroys, or truncates only the mutated form of a gene or a fragment (e.g., a mutant allele), and leaves the wild type form (e.g., a wildtype allele) intact, the donor template or wild type gene sequence that is supplemented to the cells or a patient may not be codon-modified. Under such circumstances, the DNA digesting agent (e.g., gRNA or sgRNA in combination with a Cas enzyme) would be designed to recognize and target only the mutated form of a disease-related gene (and not recognize and target a wild type form of the gene).

The donor/template polynucleotide may be integrated into the endogenous gene. Such targeted integration may be accomplished by homologous recombination. In one embodiment, the donor/template polynucleotide is flanked by an upstream and a downstream homology arm. The homology arms, which flank the donor sequence, correspond to regions within the targeted locus. Alternatively, the donor/template polynucleotide (whether codon-modified or not) of a gene of interest or fragment is not integrated into the endogenous gene. The donor/template polynucleotide may offer expression without integration into the host genome.

### Diagnostic Agents

The cargo or payload may comprise a diagnostic agent including, but not limited to, radioactive tracers or radionuclides used for positron emission tomography (PET) and other scans, including, but not limited to, carbon-11, nitrogen-13, oxygen-15, and fluorine-18. These agents can be more precisely delivered to the tissue of interest by loading them on an engineered exosome which either artificially or naturally targets the tissue of interest, i.e., the tissue that is being scanned, thus, reducing off-target delivery of radioisotopes.

### Methods of Use

The present application discloses methods of increasing delivery vehicle uptake by cells. In some embodiments, the methods are used for the improved delivery of delivery vehicles, including exosomes, and their cargoes, such as small molecules, RNAs, non-coding RNAs, DNA, and siRNA. In some embodiments, the methods are used for increasing uptake of therapeutic exosomes by priming cells with ATP-loaded exosomes. In some embodiments, the methods are used for increasing endocytosis for nanoparticle uptake using ATP-loaded exosomes. In some embodiments, the methods are used for delivering ATP to the cells to reprogram their metabolomic using ATP-loaded exosomes. In some embodiments, the methods are used for co-delivering ATP with other therapeutics to increase higher therapeutic index.

### Pharmaceutical Compositions

The present disclosure provides a pharmaceutical composition comprising the present delivery vehicles. In some embodiments, the present delivery vehicles may be mixed with a pharmaceutically acceptable carrier to form a pharmaceutical composition. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of a disease or disorder in the subject. In some embodiments, the subject is a human.

Pharmaceutically acceptable carriers and excipients are well known in the art, and may comprise, for example, buffers including phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants.

The present delivery vehicles may be delivered to a cell by contacting the cell with the delivery vehicles. The present delivery vehicles or the present composition may be delivered/administered to a subject by any route, including, but not limited to, intravenous, intracerebroventricular (ICV) injection, intracisternal injection or infusion, oral, transdermal, ocular, intraperitoneal, subcutaneous, implant, sublingual, subcutaneous, intramuscular, rectal, mucosal, ophthalmic, intrathecal, intra-articular, intra-arterial, sub-arachnoid, bronchial and lymphatic administration. The present composition may be administered parenterally or systemically. The present composition may be administered locally.

Intravenous forms include, but are not limited to, bolus and drip injections. Examples of intravenous dosage forms include, but are not limited to, water for injection USP; aqueous vehicles including, but not limited to, sodium chloride injection, Ringer's injection, dextrose injection, dextrose and sodium chloride injection, and lactated Ringer's injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

### EXAMPLES

This invention will be better understood from the experimental details which follow. However, one skilled in the art will readily appreciate that the specific methods and results discussed are merely illustrative of the invention as described more fully in the claims that follow thereafter.

### Example 1: Effect of Loading ATP (exoBOOST) on Target Cell Exosome Uptake

Fluorescently-labeled exosomes (100 μg) devoid of nucleic acids as described in PCT application no. PCT/US2020/047894 (safeEXO), which is hereby incorporated by reference in its entirety, were loaded with 5 mM ATP using electroporation (300 V, 150 UF) with appropriate controls. Control labeled exosomes were either mock electroporated or co-incubated with ATP without electroporation. ATP-eletroporated exosomes, exosomes incubated with ATP, and control exosomes were subsequently re-isolated using ExoQuick-TC™ brand reagent (commercially available from System Biosciences of Palo Alto, California) and washed and cocultured with epithelial cells. After 90 minutes, the media was removed and the cells were washed two times with PBS, and the cells were then imaged using a ZOE™ Fluorescent Cell Imager, manufactured by Bio-Rad® Laboratories. The images show a significantly increased exosome uptake in ATP-loaded exosomes (FIG. 1A). Quantification of these results demonstrated a significant, greater than 300% increased uptake with ATP-loaded exosomes compared to non-electroporated exosomes. The results shown in FIG. 1B are representative of three independent experiments.

Additionally, 100 μg of exosomes (as described in PCT application no. PCT/US2020/047894) (safeEXO) were loaded with ATP in an independent manner using the Exo-Fect™ siRNA/miRNA Transfection Kit reagent, manufactured by System Biosciences®, based on the manufacturer's protocol. Similar to electroporation, the results demonstrated a significant, greater than 300% increased uptake with ATP-loaded exosomes when compared to the control. The results of FIG. 1C are representative of three independent experiments.

### Example 2

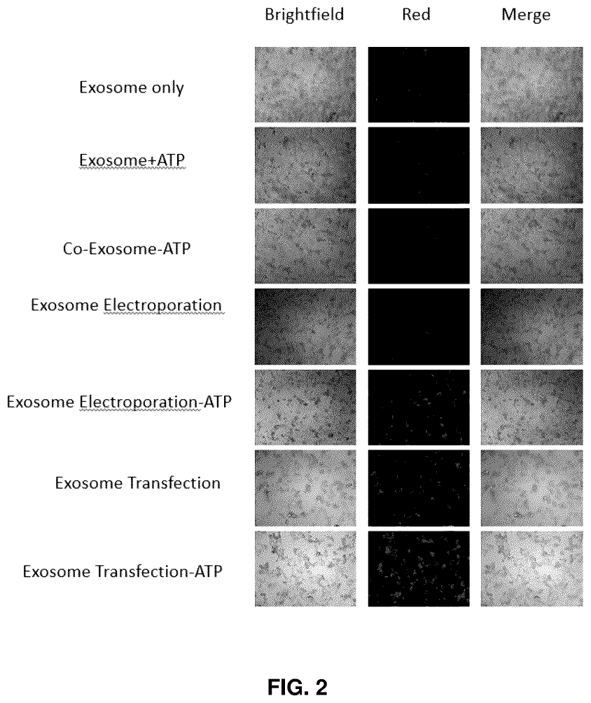

The safeEXO exosomes were transfected with an exosome transfection reagent from the Exo-Fect™ siRNA/miRNA Transfection Kit, manufactured by System Biosciences®, following the manufacturer's protocol, or was electroporated with ATP, or co-incubated with 10 mM ATP. Further, safeEXOs were labeled PKH26 red fluorescent dye (Sigma® #MINI26-1KT) according to the manufacturer's protocol and were further filtered through a 100 kda filter to remove any further suspended dye. KRAS 4B wild type cells were then seeded in 48 wells (40,000 cells per well) and were treated with PKH26 red fluorescent labeled ATP transfected or untransfected safeEXO. After 2 hours, the wells were washed twice with 1×PBS and were imaged using a ZOE™ Fluorescent Cell Imager, manufactured by Bio-Rad® Laboratories. The images are shown in FIG. 2.

### Example 3

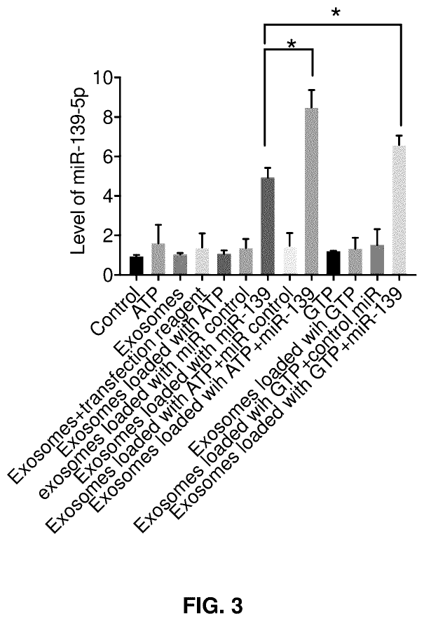

FIG. 3 shows that loading exosomes with ATP and GTP increases their uptake and cargo delivery. 100 μg of exosomes were loaded with ATP (5 mM), miR-139-5p (25 nM), GTP (5 mM), combinations of those, or a negative control (25 nM), as indicated by transfection reagents (Exo-Fect™). The levels of miR-139-5p were quantified by a TaqMan microRNA assay. The level of miR-139-5p was normalized based on RNU48. The “*” in FIG. 3 indicates a p value<0.05.

It is to be understood that the enhanced delivery vehicle for a treating agent is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. An enhanced delivery vehicle for a treating agent, comprising:
a base delivery vehicle adapted for carrying a treating agent for uptake thereof by a target cell; and
an enhancing component selected from the group consisting of at least one energy rich substance, at least one agent which increases production of at least one energy rich substance in the target cell, and a combination thereof,
whereby the enhancing component enhances the uptake of the base delivery vehicle by the target cell.

2. The enhanced delivery vehicle as recited in claim 1, wherein the base delivery vehicle is selected from the group consisting of exosomes, microvesicles, apoptotic bodies, oncosomes, extracellular vesicles, microparticles, liposomes, and nanoparticles.

3. The enhanced delivery vehicle as recited in claim 1, wherein the enhancing component comprises an exosome engineered to be substantially devoid of endogenous nucleic acids.

4. The enhanced delivery vehicle as recited in claim 1, wherein the enhancing component comprises the at least one energy rich substance, the at least one energy rich substance being selected from the group consisting of ATP, NADH, NADPH, FADH2, acetyl CoA, glucose, pyruvate, GTP, CTP, and UTP.

5. The enhanced delivery vehicle as recited in claim 1, further comprising additional cargo.

6. The enhanced delivery vehicle as recited in claim 5, wherein the additional cargo is selected from the group consisting of therapeutic agents, diagnostic agents and a combination thereof.

7. The enhanced delivery vehicle as recited in claim 5, wherein the additional cargo is selected from the group consisting of a nucleic acid, a protein, a polypeptide, a small molecule and combinations thereof.

8. The enhanced delivery vehicle as recited in claim 5, wherein the additional cargo comprises a nucleic acid selected from the group consisting of a single-stranded DNA (ssDNA), a double-stranded DNA (dsDNA), a donor/template DNA, s cDNA, a DNA encoding one or more RNAs, a sgRNA, a guide RNA (gRNA), a prime editing guide RNA (pegRNA), a microRNA (miRNA) inhibitor, a miRNA mimic, a small interfering RNA (siRNA), small synthetic RNA, a synthetic RNA, an antisense oligonucleotide, a short hairpin RNA (shRNA), a double-stranded RNA (dsRNA), an antisense RNA, a ribozyme, and combinations thereof.

9. The enhanced delivery vehicle as recited in of claim 5, wherein the additional cargo comprises an endonuclease selected from the group consisting of a zinc finger nuclease (ZFN), a ZFN dimer, a ZFNickase, a transcription activator-like effector nuclease (TALEN), a meganuclease, and an RNA-guided DNA endonuclease.

10. The enhanced delivery vehicle as recited in claim 5, wherein the additional cargo comprises an RNA-guided DNA endonuclease comprising a Clustered Regularly Interspaced Short Palindromic Repeats (CRISPR)-CRISPR associated (Cas) (CRISPR-Cas) system.

11. A method of delivering a treating agent to a target cell, comprising contacting a target cell with an enhanced delivery vehicle carrying at least one treating agent, wherein the enhanced delivery vehicle comprises:
a base delivery vehicle adapted for carrying the at least one treating agent for uptake thereof by the target cell; and
an enhancing component selected from the group consisting of at least one energy rich substance, at least one agent which increases production of at least one energy rich substance in the target cell, and a combination thereof,
whereby the enhancing component enhances the uptake of the base delivery vehicle by the target cell.

12. The method of delivering a treating agent to a target cell as recited in claim 11, wherein the base delivery vehicle is selected from the group consisting of exosomes, microvesicles, apoptotic bodies, oncosomes, extracellular vesicles, microparticles, liposomes, and nanoparticles.

13. The method of delivering a treating agent to a target cell as recited in claim 11, wherein the enhancing component comprises an exosome engineered to be substantially devoid of endogenous nucleic acids.

14. The method of delivering a treating agent to a target cell as recited in claim 11, wherein the enhancing component comprises the at least one energy rich substance, the at least one energy rich substance being selected from the group consisting of ATP, NADH, NADPH, FADH2, acetyl CoA, glucose, pyruvate, GTP, CTP, and UTP.

15. The method of delivering a treating agent to a target cell as recited in claim 11, wherein the enhanced delivery vehicle further comprises additional cargo.

16. The method of delivering a treating agent to a target cell as recited in claim 15, wherein the additional cargo is selected from the group consisting of therapeutic agents, diagnostic agents and a combination thereof.

17. The method of delivering a treating agent to a target cell as recited in claim 15, wherein the additional cargo is selected from the group consisting of a nucleic acid, a protein, a polypeptide, a small molecule and combinations thereof.

18. The method of delivering a treating agent to a target cell as recited in claim 15, wherein the additional cargo comprises a nucleic acid selected from the group consisting of a single-stranded DNA (ssDNA), a double-stranded DNA (dsDNA), a donor/template DNA, s cDNA, a DNA encoding one or more RNAs, a sgRNA, a guide RNA (gRNA), a prime editing guide RNA (pegRNA), a microRNA (miRNA) inhibitor, a miRNA mimic, a small interfering RNA (siRNA), small synthetic RNA, a synthetic RNA, an antisense oligonucleotide, a short hairpin RNA (shRNA), a double-stranded RNA (dsRNA), an antisense RNA, a ribozyme, and combinations thereof.

19. The method of delivering a treating agent to a target cell as recited in of claim 15, wherein the additional cargo comprises an endonuclease selected from the group consisting of a zinc finger nuclease (ZFN), a ZFN dimer, a ZFNickase, a transcription activator-like effector nuclease (TALEN), a meganuclease, and an RNA-guided DNA endonuclease.

20. The method of delivering a treating agent to a target cell as recited in claim 15, wherein the additional cargo comprises an RNA-guided DNA endonuclease comprising a Clustered Regularly Interspaced Short Palindromic Repeats (CRISPR)-CRISPR associated (Cas) (CRISPR-Cas) system.
